(19) United States
(12) Patent Application Publication
(10) Pub. No.: US 2016/0279116 A1
(43) Pub. Date: Sep. 29, 2016

(54) THERAPEUTIC USES OF NICOTINE

(71) Applicant: Arturo SOLIS HERRERA, Aguascalientes (MX)

(72) Inventor: Arturo SOLIS HERRERA, Aguascalientes (MX)

(21) Appl. No.: 15/176,555

(22) Filed: Jun. 8, 2016

Related U.S. Application Data
(63) Continuation of application No. 14/860,973, filed on Sep. 22, 2015.
Application No. 14/860,973 is a division of application No. 13/534,710, filed on Jun. 27, 2012, now abandoned.
Application No. 13/534,710 is a division of application No. 12/418,993, filed on Apr. 6, 2009, now abandoned.
Application No. 12/418,993 is a continuation-in-part of application No. PCT/MX2006/000031, filed on May 8, 2006.

(51) Int. Cl. A61K 31/465; A61K 31/7036; A61K 31/616; A61K 9/00; A61K 9/08

(52) U.S. Cl. CPC A61K 31/465; A61K 9/006; A61K 9/08; A61K 31/616; A61K 31/7036

(57) ABSTRACT

The use of nicotine for treating renal pain is described. It is believed that renal pain, such as renal pain caused by renal ischemia or renal reperfusion, can be improved by increasing the activity of α-melanocyte stimulating hormone (α-MSH), the release of which is affected by nicotine.

### CROSS-REFERENCE TO RELATED APPLICATION

This application is a continuation of U.S. application Ser. No. 14/860,973, filed Sep. 22, 2015, which is a division of U.S. application Ser. No. 13/534,710, filed Jun. 27, 2012, now abandoned, which is a division of U.S. application Ser. No. 12/418,993, filed Apr. 6, 2009, now abandoned, which is a continuation-in-part of Patent Cooperation Treaty Application PCT/MX2006/000031, filed May 8, 2006, and the disclosures of all of the prior applications are incorporated by reference herein in their entireties.

### OBJECTIVE OF THE INVENTION

This invention protects the use of substances, such as nicotine, analogues, precursors or derivatives thereof, that promote, facilitate or intensify the releasing and the action or activity of α-MSH (alpha-melanocyte stimulating hormone), through their indirect effect mainly on the melanotrophs located in the pars intermedia of the hypophysis in close relationship with lactotrophs.

There are different susceptible pathological conditions which can be improved by administration of α-MSH, because the stem cells that respond to the stimulus participate in several main functions in the organism. Examples of such pathological conditions include, but are not limitated to: proliferative retinopathy where the eye fibroblast, as any organism fibroblast, in the presence of hypoxia reacts with secreting collagen (Dr. Humberto Montoya de Lira, 2000); initial stages of retrolental fibroplasia; the proliferative diabetic retinopathy; the post-traumatic proliferative retinopathy: primary, secondary, local and distant; the proliferative retinopathy caused by hypoxia: primary, secondary, local and distant; infectious syndrome where secondary alterations of liver, kidney and lung may be prevented; conditions where α-MSH is a protective factor against the conditions, such as the degenerative osteoarthritis, eclampsia, Parkinson's disease, Alzheimer's disease, arthritis from different etiologies, the rejection of transplanted tissues. In addition, α-MSH improves depression; diminishes 95% of the tissue deterioration in experimental models of ischemia/reperfusion in kidney, lung, intestine; protects vessels from deterioration caused by bacterial LPS (lipopolysaccharides); and protects liver from deterioration induced by LPS. The α-MSH has also been reported to diminish liver cirrhosis; at the same time α-MSH is considered a protective factor in degenerative osteoarthritis, it seems to protect cartilage. Also antidepressive effect has been described for α-MSH, which can have an important therapeutic role in obesity control.

### BACKGROUND OF THE INVENTION

The α-MSH is a three decapeptide with a potent anti-inflammatory action, with prominent actions in reducing the inflammatory mediators, for example. It reduces the level of tumor necrosis factor, including cytokines. Alpha-MSH is a compound of 13 amino acids derivated from propiomelanocortin, it expresses in several regions of the Central Nervous System and in peripheral cells, including melanocytes, phagocytes, macrophages, chondrocytes, keratocytes, glial cells and keratinocytes among other stem cells. Up to date there have not been identified all the stem cells that respond to α-MSH.

The anti-inflammatory effects of α-MSH are mainly through the antagonism of proinflammatory mediators including α-tumor necrosis factor (alpha-TNF), interleukin 6 (IL-6) and nitric oxide (NO), but its powerful actions are very constant in all tissues and inclusive they superimpose. The α-MSH neuropeptide is an endogenic modulator of inflammation. The idea that α-MSH is important in the host responses begins from the initial observation from the antipyretic properties of the molecule. The α-MSH's potency for reducing the fever resulted from endogenous pyrogens is dramatic: 20,000 (twenty thousand) times as great as acetaminophen (Airagui, Lorena 2000) when the relation molecule to molecule is compared. Alpha-MSH also inhibits fever caused by endotoxin, IL-6 and alpha-TNF (α-TNF). It has an inhibitor effect on IL-1, and on the increase induced by α-TNF in the circulating proteins from acute stage and neutrophils. The α-MSH also inhibits the tissue trauma in systemic inflammation models as acute respiratory syndrome and peritonitis caused by cecal ligation and puncture as well as in ischemic acute renal defect.

Mortality, by combination of acute renal insufficiency and acute respiratory insufficiency, reaches 80%. The severe trauma, burns, hemorrhages, sepsis, shock, or severe local tissue trauma, can initiate a systemic inflammatory response provoking the multiple organic failure and death. There are pathogenic and epidemiological connections between renal and lung trauma. A great part of risk increase due to acute renal failure after heart surgery comes from extra renal complications such as respiratory failure.

Severe tissue trauma happened after a prolonged ischemia in the inferior torso or during a complicated surgery of abdominal aortic aneurysms or during an acute respiratory failure syndrome.

In animal models, secondary (or distant) pulmonary trauma can be started by severe local ischemia in liver, the gastrointestinal tract, inferior member, kidney or chemical pancreatitis. For example, renal traumatism by ischemia/reperfusion, can increase lung vascular permeability, as well as produce interstitial edema, alveolar hemorrhage and damage of rheological properties of erythrocytes. Because lung has the biggest microcapilar trauma in the organism, it responds to circulating proinflammatory signs with activation of lung macrophages, secretion of proinflammatory cytokines, attraction of neutrophils and macrophages, finally resulting a lung trauma.

There are many similarities between the activations of local pathways of tissue trauma after pulmonary trauma and acute renal and secondary pulmonary traumatism. The renal ischemia/reperfusion causes apoptosis and necrosis in rectum proximal tubules and inflammatory infiltration of leukocytes. Earlier in the reperfusion period, there is an activation of activated kinases by stress (for example, kinase protein p-38 activated by mitogens [MAPK]), and by transcription KB factor of nuclear factors (NF-κB) and protein activator (AP-1) and induction of proinflammatory cytokines (α-TNF) and adhesion molecules (intercellular adhesion molecule-1 [ICAM-1]). The selective inhibition of α-TNF and/or of ICAM-1 diminishes acute renal trauma. In paralle, proinflamatory traces NF-κB, p-38 and AP-1 are activated after acute pulmonary trauma. The inhibition of NF-κB and p-38 reduce distant (or secondary) pulmonary trauma. But, there is no agent that has been shown to inhibit both local trauma and distant pulmonary trauma. For example the inhibitor of p-38 CNI-1493 partially reduces distant pulmonary trauma but does not have effect in subjacent renal trauma by ischemia/reperfusion.

Alpha-MSH hormone is an anti-inflammatory cytokine that inhibits chronic or acute systemic inflammation. Alpha-MSH inhibits renal trauma by ischemia/reperfusion, by cisplatin administration, or after a transplant by marginal donor; but not after administration of mercury (mercury poisons melanocytes).

Mechanism of action of α-MSH is extensive, and the actions documented by us are: the inhibition of inflammatory traces, cytotoxic and apoptotic pathways activated by renal ischemia.

It has been demonstrated that α-MSH inhibits activation of α-TNF and ICAM-1 four hours after the reperfusion. Although, the earlier molecular mechanisms activated by α-MSH are not elucidated. In models of ischemia/reperfusion disease and other similar models, α-MSH inhibits the production of many cytokines, chemokines, and the inducible synthase of nitric oxide. This suggests that α-MSH acts in one or several early common steps in initial pathway of inflammation. Recent studies have demonstrated that α-MSH suppresses the stimulation of NF-κB in brain inflammation and in cell culture exposed to LPS.

Alpha-MSH also inhibits p38 MAPK in melanoma cells B16 and in AP-ligand-DNA in dermal fibroblasts, but not in macrophages. By means, it has been determined that α-MSH diminishes pulmonary trauma caused by renal trauma from ischemia/reperfusion.

In animal models, it has been demonstrated that serum creatinine increases, in an important form, at 4, 8 and 24 hours after renal ischemia/reperfusion in comparison with witness and control animal. At the same time, animals that received α-MSH had important lower levels of creatinine than animals that only had vehicles as well as less cylinders and necrosis evaluated by quantitative cytology at 4 hours.

Effects of α-MSH in leukocyte accumulation: Preliminary studies have shown that renal ischemia causes leukocyte infiltration in kidney and lung and α-MSH inhibits locally leukocyte accumulation after acute inflammation and in renal ischemia. The stain with stearate of chloroacetate shows an increase in leukocyte accumulation in lung four hours after renal ischemia/reperfusion compared with control animals. Treatment with α-MSH before releasing of patch (clamp) diminishes leukocyte infiltration. These changes were evaluated by counting positive stearase cells in lung and kidney. There were elevated infiltrating leukocytes in lung and kidney in very early stages after renal trauma by ischemia/reperfusion, and said accumulation was inhibited by α-MSH.

Effects of α-MSH on α-TNF and ICAM-1. Renal ischemia increases α-TNF and ICAM-1 and the inhibition of both pathways diminishes, in a dramatic form, the renal damage. It has been found that renal ischemia causes phosphorylation (by means, activation) from IκBα cytosolic in kidney as well as in lung during 15-30 minutes after reperfusion. Administration of α-MSH just before releasing of patch (clamp) inhibits phosphorylation of IκBα as in kidney and lung.

Phosphorylation of IκBα causes its own destruction; it allows that the dimers of NF-κB containing p65 translocated to the nucleus. As it was to hope, phosphorylation of IκBα rapidly induces the appearance of p65 in the nucleus in kidney as well as in lung, which could be inhibited by administration of α-MSH.

The activity of ligand NF-κB increased rapidly in lung as well as in kidney at the end of the ischemia period. The treatment with α-MSH inhibited the ligand NF-κB activity in kidney and lung.

Renal ischemia/reperfusion also causes a rapid phosphorylation (and of course activation) from p38 of kidney and lung without changes in the total p38. Phosphorylation of p38 was inhibited with α-MSH treatment.

It has been found inflammatory cells infiltrating, intensely and rapidly, the lung after renal ischemia. It has been proven that α-MSH has a dramatic effect in pulmonary trauma, because it inhibits pulmonary infiltration in 4 and 8 hours after renal ischemia, with similar effects on kidney. Effect of α-MSH is more dramatic at 8 hours than in 4 hours, may be because it can inhibit most early responses of stress/inflammations, some or all of them can contribute to the ability of α-MSH for diminishing the progress of damage.

It has been found that renal ischemia/reperfusion increases levels of mRNA (messenger) for α-TNF and ICAM-1 after cisplatin inhibition.

Alpha-TNF is important in pathogenesis of distant organ damage, because antibodies against α-TNF reduce pulmonary damage after liver ischemia and agents that diminish distant pulmonary damage also diminish α-TNF located in pulmonary tissue. This evidence suggests the importance of inflammation and α-TNF particularly in distant pulmonary trauma induced by ischemia or damage to extra pulmonary organs.

Some of α-MSH effects are probably mediated by direct effect on leukocytes, because the neutrophils and macrophages express receptors for α-MSH.

Alpha-MSH inhibits the migration of neutrophils in vitro and the production of nitric oxide in culture of macrophages. The α-MSH inhibits damage by renal ischemia/reperfusion until in absence of leukocyte infiltration, which suggests that α-MSH can also act by different ways from leukocytes.

Administration of α-MSH just before reperfusion has a great protective effect in lung as well as in kidney. Alpha-MSH reduces, in a dramatic form, the activation of distant or secondary pulmonary damage caused by lung transplant, pancreatitis, liver ischemia, hemorrhages or secondary reactions to bacterial lipopolysaccharides.

The events that cause distant renal damage after renal ischemia/reperfusion are unknown.

Pretreatment for distant ischemia that inhibits terminal-kinase C-Jun N and activation of p38, prevents renal damage after ischemia/reperfusion. But unfortunately, it is not a viable therapeutic alternative. The administration of α-MSH is much more practical.

There is more evidence every time that suggests the activation of NF-κB proceeds and may cause secretion of α-TNF after myocardial ischemia and kidney ischemia.

The combined acute lung and kidney failure comes with an extremely high morbidity and mortality, whose subjacent mechanisms are unknown but administration of α-MSH improves over life in 90%. Alpha-MSH reverts liver cirrhosis, gets better treatment of diseases such as Alzheimer's disease, prevents Parkinson's disease, among many others. The severe tissue trauma presented in the burns, in the polytraumatized patients, in the prolonged ischemia of the inferior torso or complicated surgery for abdominal aortic aneurysms, frequently provokes the subsequent events that cause the multiple organic failures. Actually therapeutic steps available are very elemental and are limited to replacing the function of the lost organ, controlling ventilation and dialysis, preventing barotraumas, and optimizing cardiovascular function with resuscitation of the adequate volume and inotropic support. Treatment with medication is not desirable. Recently, C-protein activated showed some utility to diminish death by sepsis. Additional strategies to prevent and/or treat multiple organ failures will be extremely useful. In this moment we do not know any medicine that reduces pulmonary damage nor renal damage.

It has been demonstrated that administration of α-MSH just before reperfusion inhibits acute renal damage as well as pulmonary damage.

The ability of α-MSH to inhibit the damage in both organs, the extension of protection that reaches or gets, and the wide action mechanism distinguishes α-MSH from other agents used to prevent, limit, protect or delay damage by ischemia/reperfusion. This suggests that α-MSH can have an important therapeutic effect on adequate patients. (Deng, Hu, Yuen, Star, Am J Respir Crit Care Med Vol 169 pp 749-756, 2004.).

Alpha-MSH can have an important therapeutic role for treatment of vasculitis, sepsis, chronic and acute inflammatory diseases from different etiologies. (Endocrinology 144: 360-370, 2003).

In intermittent hemodialysis, it can characteristically appreciate elevation of α-TNF, IL-6 and NO, so that α-MSH is liberated in these patients to counteract the proinflammatory effects of these cytokines (Lorena Airagui, Leticia Garofalo, Maria Grazia Cutuli. Nephrol Dial Trans 2000 (15):1212-1216).

Alpha-MSH modulates α-TNF locally and circulating in experimental models of brain inflammation (Nilum Rajora, Giovanni Boccoli, Dennos Burns. The Journal of Neuroscience Mar. 15, 1997; 17(6): 2181-2186.) In this research, the secretion of α-TNF in central nervous system was induced by a local injection of bacterial LPS. The plasma concentration of α-TNF had an important elevation after central application of LPS, indicating that the host peripheral response was increased by induction of CNS signaling.

The inhibition of α-TNF synthesis by α-MSH was confirmed using inhibition of mRNA. Although some inflammatory cytokines contribute to Central Nervous System (CNS) inflammation, α-TNF is specially important because it is identified as an important agent in physiopathogenicity of CNS diseases as multiple sclerosis, HIV infection of CNS, Alzheimer's disease, meningitis, severe cranioencephalic trauma consequential to the ischemia/reperfusion and/or trauma. The increase of α-MSH levels, by endogenic or exogenic administration, has an important therapeutic or prophylactic effect for diminishing the diseases with α-TNF increased, as above mentioned.

Effect of α-MSH in Central Nervous System (CNS) degeneration. Almost every one of degenerative diseases of CNS is associated with chronic inflammation. An important stage in this process is the activation of brain phagocytic mononuclear cells named microglia. Nicotine neuoprotector effects due to its action over selective nicotinic antagonist receptors α7 in illnesses such as Parkinson's disease, Alzheimer's disease, depression, obesity, aging, etc., have been reported (R Douglas Shytle, Takashi Mori, Kira Townsed, Cholinergic modulation of microglial activation of α-7 nicotinic receptors. Journal of Neurochemistry , 2004, 89, 337-443.)

It is congruent that beneficial effects of α-MSH include whole organism such as skin, mucus, eyes, intestine, muscle, joints, etc, because they have common metabolic pathways stimulated by the hormone.

### DETAILED DESCRIPTION OF THE INVENTION

The invention mainly consists in administration of nicotine, analogues, precursors or derivatives thereof to adequate patients, in pharmacophores and effective dosage, by the suitable pathway in each case, in therapeutic form and/or prophylactic form. Through its effect on hypothalamus (main action but not the unique), the α-MSH releasing is induced by melanotrophs from pars intermedia of the hypophysis, because this secretion (α-MSH) is tonic. By such means the hypothalamus has a suppressor effect more than secretor, to differentiate from others hypothalamic effects on hypophysis. It seems to be one of the few hypophysary hormones released in tonic form (constant) and the hypothalamus inhibits this releasing through the dopamine secretion (hypothalamic) (another hypophysary hormone released in tonic form is the prolactin from mammotrophs). The nicotine, precursors, analogues or derivatives thereof, administrated by adequate form in effective dosage and adequate pharmacophore, provokes an effect on hypothalamus, diminishing dopamine secretion, and the melanotrophs of the pars intermedia of the hypophysis releases α-MSH tonically (the more hypothalamic inhibition, the less tonic α-MSH release, and vice versa), as it happens during all of its life, as a result of several factors. The secretion of dopamine is diminished and/or almost completely interrupted. The several factors can be environmental, emotional, different acute or chronic diseases, infectious diseases, surgeries, different therapeutic actions, pesticides, hormones, chemical agents, different xenobiotic types, etc., which incite the α-MSH secretion in all cases, in one or another sense. The action of nicotine suggested herein may not be only the unique effect of nicotine, analogues, precursors or derivatives thereof. The action is to provoke the α-MSH releasing mainly from melanotrophs located in the pars intermidia of the hypophysis in close contact with mammotrophs. Although it is not the unique pathway that may be documented in complete and more scientific form, up to now it is the only way in which we can document in the more complete and scientific form, without ruling out other action sites in the skin (keratinocytes), pilose follicles, etc., macrophages, etc. The action may be depending on the nicotine dosage used as well as the administration pathway.

This present invention relates to pharmaceutical compositions with active substances and pharmaceutical vehicles that induce releasing of endogenic α-MSH in humans, coming from stem cells. These compositions can have prophylactic and/or therapeutic purposes in inflammatory chronic and/or acute, degenerative and infectious diseases.

The releasing of α-MSH provokes the “photosynthesis” in human (patient) and animal, because the release of α-MSH increases the synthesis of melanin, which promotes the releasing of oxygen and hydrogen in the tissue from water (WO2006/132521), increasing importantly the energy available to eukaryotic cells and energizing the main reactions during the life. This energy, which is estimated in a third part, is used or required, of the whole. It is not additional, moreover it is the mainly, which must happen at the first time, in order to provoke other ones. Its diminishing provokes that the other two third parts will also be reduced, promoting disease. This explains why the photosynthesis stimulates the induction of endogenic α-MSH release, (by administration of nicotine, its derivatives or its analogues), provokes a dramatic positive response in all tissues. It is difficult to understand how nicotine, its derivatives or analogues provoke so many good effects in all tissues.

NASA defines life as a self sustainable chemical system that eventually is in Darwinian evolution. Melanin may be precursor of life because it is stable in water, and could have been stood in it during thousands of years and more. In water, with electromagnetic radiations originated from sun, melanin generated energy in almost constant form. It was across the time for provoking the other chemical reactions done by the first living organisms, because it disposed of elemental energy for the beginnings of chemical system that after was completed with carbon sources such as glucose-6-phosphate, but which were only afterwards. We could say that melanin is to animal kingdom as chlorophyll is to vegetal kingdom.

About the use of nicotine according to embodiments of the invention we have some examples.

### EXAMPLE 1

Female patient 27 years old, she was in the ninth month of pregnancy without diabetes or hypertension or neuropathy antecedents. There was no surgery antecedent. It began with an intense pain in the right renal region in 72 hours of evolution, she could not sleep, required the administration of analgesics every three hours. Twenty four hours later amikacin IM was administered every 12 hours. She could not be in a free attitude due the intense pain, apart from the natural upsets in the ninth month of pregnancy. It was decided to administer nicotine in watery vehicle by sublingual pathway in a concentration of 3 mg/ml.

At the beginning 15 drops were administrated and 30 minutes later 10 drops more. Patient slept and after three days she could sleep all night, pain diminished significantly that she did not awake. General physical state improved in the dramatic form, the analgesic was limited to a half aspirin every 12 hours and the antibiotic course continued for 8 days more. Nicotine was administrated during 4 weeks in dosage of 5 drops by sublingual pathway every three hours.

### EXAMPLE 2

Male patient just born, (his mother is patient from example 1) born by cesarean who had in the first hours hypothermia and vomit, few hours after petechiae appeared in the back. Plateletopenia was found from blood analysis and increase of sedimentation velocity analysis. Considering that it was a sepsis, amikacin IV was administered initially. Agree to mother treatment, it was began the administration of nicotine by sublingual pathway in a dosage of 1 drop every 12 or 24 hours; in concentration of 3 mg/mL. Patient slept deep and long, curiously the heart increased its rate from 110 per minute to 130 and the peripheral oxygen was not diminished of 93%. Twenty four hours later the baby had increased 80 grams of weight. Now the kid is growing well and without consequences.

### EXAMPLE 3

Male patient 25 years old with post traumatic bleeding (hyphema). He was reviewed at 14^{th} day of the disease, and the hyphema of 90% did not improve with the first treatment. The patient came to us because his doctor suggested him a surgery to evacuate blood for avoiding losing his eye. We explained to the patient the treatment to stimulate α-MSH could be an alternative form in order to protect the tissue from apoptosis as a potent anti-inflammatory agent, when the nicotine induces the α-MSH release. We indicated a dosage of 2 drops sublingual pathway every hour, for the hyphema was of 90% the vision was poor and the intraocular pressure was 40 mm Hg despite last treatment. All medicine was suspended and began the new treatment. Three weeks later vision was 20/40. The recovery was dramatic and complete in 90% after four weeks.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A method of treating renal pain caused by renal ischemia in a human subject in need thereof, comprising sublingually administering to the human subject a pharmaceutical composition comprising a pharmaceutically acceptable vehicle and an effective amount of nicotine, such that the renal pain is treated.

2. The method of claim 1, wherein the composition is an aqueous pharmaceutical composition.

3. The method of claim 1, wherein the pharmaceutical composition is an aqueous pharmaceutical composition consisting of water and the effective amount of nicotine.

4. The method of claim 1, wherein the effective amount of nicotine in the pharmaceutical composition is 3 mg/ml.

5. The method of claim 3, wherein the effective amount of nicotine in the pharmaceutical composition is 3 mg/ml.

6. The method of claim 1, wherein the composition is administered to the human subject at least once per day for a duration of at least 4 weeks.

7. The method of claim 1, further comprising administering an analgesic or antibiotic.

8. A method of treating renal pain caused by renal reperfusion in a human subject in need thereof, comprising sublingually administering to the human subject a pharmaceutical composition comprising a pharmaceutically acceptable vehicle and an effective amount of nicotine, such that the renal pain is treated.

9. The method of claim 8, wherein the composition is an aqueous pharmaceutical composition.

10. The method of claim 8, wherein the pharmaceutical composition is an aqueous pharmaceutical composition consisting of water and the effective amount of nicotine.

11. The method of claim 8, wherein the effective amount of nicotine in the pharmaceutical composition is 3 mg/ml.

12. The method of claim 10, wherein the effective amount of nicotine in the pharmaceutical composition is 3 mg/ml.

13. The method of claim 8, wherein the composition is administered to the human subject at least once per day for a duration of at least 4 weeks.

14. The method of claim 8, further comprising administering an analgesic or antibiotic.

15. A method of treating renal pain caused by renal ischemia or renal reperfusion in a human subject in need thereof, comprising sublingually administering to the human subject an aqueous pharmaceutical composition comprising a pharmaceutically acceptable vehicle and an effective amount of nicotine at least once per day for a duration of at least 4 weeks, such that the renal pain is treated.

16. The method of claim 15, wherein the aqueous pharmaceutical composition consists of water and an effective amount of nicotine.

17. The method of claim 15, wherein the effective amount of nicotine in the pharmaceutical composition is 3 mg/ml.

18. The method of claim 15, wherein the subject is in need of treatment of renal pain caused by renal ischemia.

19. The method of claim 15, wherein the subject is in need of treatment of renal pain caused by renal reperfusion.

20. The method of claim 15, further comprising administering an analgesic or antibiotic.
